(19) United States
(12) Patent Application Publication
(10) Pub. No.: US 2021/0052474 A1
(43) Pub. Date: Feb. 25, 2021

(54) SUNSCREEN COMPOSITION

(71) Applicant: Hyuma FUJINOHARA, Tokyo (JP)

(72) Inventor: Hyuma FUJINOHARA, Tokyo (JP)

(21) Appl. No.: 16/998,447

(22) Filed: Aug. 20, 2020

(30) Foreign Application Priority Data
Aug. 22, 2019 (JP) 2019-151794

(51) Int. Cl. A61K 8/27; A61Q 17/04; A61K 8/894; A61K 8/58

(52) U.S. Cl. CPC A61K 8/27; A61Q 17/04; A61K 2800/30; A61K 8/585; A61K 8/894

(57) ABSTRACT

Provided is a sunscreen composition free of UV absorbers prohibited from being used, the sunscreen composition being ameliorated in terms of the problems with feeling of use and the problem of a white residue. The sunscreen composition includes: zinc oxide fine particles; a crosslinked polyether-modified silicone; and a silicone oil, wherein a content ratio of the zinc oxide fine particles is from 8 wt % to 40 wt %, wherein the sunscreen composition is free of titanium oxide fine particles, or contains titanium oxide fine particles at a content ratio of 0.8 wt % or less, and wherein the sunscreen composition is free of a UV absorber.

This application claims priority under 35 U.S.C. Section 119 to Japanese Patent Application No. 2019-151794 filed on Aug. 22, 2019, which is herein incorporated by reference.

### BACKGROUND OF THE INVENTION

### 1. Field of the Invention

The present invention relates to a sunscreen composition.

### 2. Description of the Related Art

A sunscreen composition generally has blended therein a UV absorber and/or a UV scattering agent as a component for protecting skin from UV light (for example, Japanese Patent Application Laid-open No. 2018-177689).

In recent years, it has been pointed out that some of the UV absorbers to be blended in sunscreen compositions (specifically, oxybenzone, octinoxate, octocrylene, and enzacamene) may adversely influence coral reefs. In some countries or regions, a decision has been made to prohibit sale or use of sunscreen compositions containing such components.

In order to obtain a high UV protection effect without using any UV absorber, a blending amount of the UV scattering agent needs to be increased. However, when the blending amount of the UV scattering agent is increased, there may arise: problems with feeling of use, such as a degradation in spread at the time of application of the sunscreen composition and a degradation in texture after the application; and a problem of a white residue, in which skin after the application becomes unnaturally white.

### SUMMARY OF THE INVENTION

The present invention has been made in order to solve the above-mentioned problems, and a primary object of the present invention is to provide a sunscreen composition free of the specific UV absorbers prohibited from being used, the sunscreen composition being ameliorated in terms of the problems with feeling of use and the problem of a white residue.

According to at least one embodiment of the present invention, there is provided a sunscreen composition, including: zinc oxide fine particles; a crosslinked polyether-modified silicone; and a silicone oil, wherein a content ratio of the zinc oxide fine particles is from 8 wt % to 40 wt %, wherein the sunscreen composition is free of titanium oxide fine particles, or contains titanium oxide fine particles at a content ratio of 0.8 wt % or less, and wherein the sunscreen composition is free of a UV absorber.

In at least one embodiment of the present invention, a content ratio of the crosslinked polyether-modified silicone is from 0.01 wt % to 1 wt %.

In at least one embodiment of the present invention, a content ratio of the silicone oil is from 5 wt % to 15 wt %.

In at least one embodiment of the present invention, the content ratio of the zinc oxide fine particles is from 15 wt % to wt %, the crosslinked polyether-modified silicone is a dimethicone/PEG-10/15 crosspolymer, a content ratio of the dimethicone/PEG-10/15 crosspolymer is from 0.01 wt % to 1 wt %, the silicone oil is dimethicone, and a blending ratio of the dimethicone is from 5 wt % to 15 wt %.

In at least one embodiment of the present invention, the sunscreen composition is substantially free of paraoxybenzoic acid alkyl esters, phenoxyethanol, and triclosan.

### DESCRIPTION OF THE EMBODIMENTS

A sunscreen composition according to at least one embodiment of the present invention contains zinc oxide (ZnO) fine particles, a crosslinked polyether-modified silicone, and a silicone oil, wherein the content ratio of the zinc oxide fine particles is from 8 wt % to 40 wt %, and wherein the sunscreen composition is free of titanium oxide (TiO₂) fine particles, or contains titanium oxide fine particles at a content ratio of 0.8 wt % or less. The sunscreen composition may further contain an oil other than the silicone oil, water, and any appropriate component depending on purposes.

In at least one embodiment of the present invention, the zinc oxide fine particles and the titanium oxide fine particles function as UV scattering agents, and in the following description, both are sometimes collectively referred to as UV scattering agent. In addition, herein, (meth)acrylic acid means acrylic acid and/or methacrylic acid. Similarly, (meth)acryloyl means acryloyl and/or methacryloyl.

The sunscreen composition is free of any UV absorber selected from oxybenzone, octinoxate, octocrylene, and enzacamene, and typically, is also free of UV absorbers other than the foregoing (e.g., cinnamic acid derivatives, such as octyl methoxycinnamate, and salicylic acid derivatives, such as octyl salicylate). The sunscreen composition according to at least one embodiment of the present invention is excellent in dispersion stability of the UV scattering agent, and hence can exhibit a sufficient UV protection effect without having any UV absorber blended therein. The sunscreen composition free of any UV absorber not only is reduced in adverse influence on coral reefs, but also can be reduced in irritation to skin.

In at least one embodiment of the present invention, the sunscreen composition is a water-in-oil emulsified composition. The use of the crosslinked polyether-modified silicone allows the zinc oxide fine particles to be extremely satisfactorily dispersed in an oil phase containing the silicone oil, and also allows a large amount of water to be retained in an internal phase. As a result, there can be obtained a sunscreen composition in which the content ratio of titanium oxide, which may cause a white residue, can be set to be equal to or lower than a predetermined value while maintaining a practically sufficient sunscreen effect, and which is excellent in feeling of use.

Now, components that may be blended into the sunscreen composition are described.

<Zinc Oxide Fine Particles>

From the viewpoints of a sunscreen effect and feeling of use, the zinc oxide fine particles to be used may have an average particle diameter of preferably from 10 nm to 200 nm, more preferably from 20 nm to 100 nm, still more preferably from 20 nm to 80 nm. In addition, when the average particle diameter is beyond the above-mentioned particle diameter ranges, the suppression of a white residue may be insufficient. The average particle diameter may be measured by any appropriate method, for example, with a laser diffraction/scattering particle size distribution analyzer.

The zinc oxide fine particles may be subjected to surface treatment from the viewpoint of improving dispersibility. In at least one embodiment of the present invention, the surface treatment may be hydrophobizing treatment.

As a method for the hydrophobizing treatment of the surfaces of the zinc oxide fine particles, there are given, for example, fatty acid treatment (e.g., isostearic acid), fatty alcohol treatment (e.g., stearyl alcohol), silicone treatment (e.g., methyl hydrogen polysiloxane or methyl polysiloxane), and fluorine treatment (e.g., a perfluoroalkyl phosphoric acid ester or a perfluoro alcohol).

The surface treatment amount in the hydrophobizing treatment may be preferably from 0.1 wt % to 10 wt %, more preferably from 1 wt % to 5 wt % with respect to the weight of the zinc oxide fine particles to be treated.

The zinc oxide fine particles may be used as they are in the form of powder, but from the viewpoint of improving dispersibility, may be used as a dispersion by being dispersed in a dispersion medium. For example, the zinc oxide fine particles subjected to the hydrophobizing treatment may be used as a dispersion by being dispersed in an oily component through use of a dispersant. Examples of the dispersant include polyhydroxystearic acid, isostearic acid, and stearic acid. In addition, examples of the oily component include a hydrocarbon oil (e.g., an isoalkane having to 16 carbon atoms, such as isodecane, isododecane, or isohexadecane), an ester oil (e.g., triethylhexanoin or a C12-15 alkyl benzoate), and a natural oil (e.g., glyceryl tri(caprylate/caprate)). The content ratio of the zinc oxide fine particles in the dispersion may be, for example, from 50 wt % to 70 wt %. A commercially available product, such as the Solaveil™ CZ series (CZ-100, CZ-200, CZ-300) manufactured by Croda, or a product available under the product name “Pulzea MZS-5104” from Suzukiyushi Industrial Corporation, maybe used as such dispersion of the zinc oxide fine particles. Of those, the Solaveil™ CZ series is preferred because the zinc oxide fine particles are controlled to have an extremely narrow particle size distribution, and are dispersed to so high a degree as to hardly form secondary particles, consequently being able to provide a sunscreen composition that has a practically sufficient UV protection effect and is excellent in dispersion stability.

The content ratio of the zinc oxide fine particles in the sunscreen composition is, for example, 8 wt % or more, preferably from 8 wt % to 40 wt %, more preferably from 10 wt % to 35 wt %, still more preferably from 11 wt % to 30 wt %, even more preferably from 15 wt % to 30 wt %, even more preferably from 15 wt % to 20 wt %. With the above-mentioned content ratio, the problem of a white residue and the problems with feeling of use can be solved while a practically sufficient UV protection effect is obtained.

<Titanium Oxide Fine Particles>

In the sunscreen composition according to at least one embodiment of the present invention, the titanium oxide fine particles are an optional component. The content ratio of the titanium oxide fine particles in the sunscreen composition is from 0 wt % to 0.8 wt %, preferably from 0 wt % to 0.5 wt %. When the content ratio of the titanium oxide fine particles is more than 0.8 wt %, a white residue may be generated.

From the viewpoints of a sunscreen effect and feeling of use, the average particle diameter of the titanium oxide fine particles is preferably from 10 nm to 200 nm, more preferably from 10 nm to 100 nm, still more preferably from 10 nm to 50 nm. The average particle diameter may be measured by any appropriate method, for example, with a laser diffraction/scattering particle size distribution analyzer.

The titanium oxide fine particles maybe subjected to surface treatment from the viewpoint of improving a UV scattering effect. Any appropriate method may be used as a method for the surface treatment of the titanium oxide fine particles. Examples thereof include: a method involving adsorbing an oil and fat onto the surface of each of the titanium oxide fine particles; fatty acid treatment; metal soap treatment; silicone treatment; and fluorine treatment. In addition, the titanium oxide fine particles may each be coated with another inorganic material to provide composite fine particles, which may then be subjected to surface treatment. Examples of the other inorganic material include silica, aluminum hydroxide, alumina, and zirconia.

The surface treatment amount of the titanium oxide fine particles is preferably from 1 wt % to 15 wt % with respect to the titanium oxide fine particles after the surface treatment.

The titanium oxide fine particles may be used as they are in the form of powder, but from the viewpoint of improving dispersibility, may be used as a dispersion by being dispersed in a dispersion medium. For example, the titanium oxide fine particles subjected to the surface treatment may be used as a dispersion by being dispersed in an oily component through use of a dispersant. Examples of the dispersant and the oily component similarly include those given as examples for the dispersion of the zinc oxide fine particles. The content ratio of the titanium oxide fine particles in the dispersion may be, for example, from 40 wt % to 60 wt %. A commercially available product, such as the Solaveil™ XT series manufactured by Croda, may be used as such dispersion of the titanium oxide fine particles.

<Crosslinked Polyether-Modified Silicone>

The crosslinked polyether-modified silicone can improve the emulsification stability of the sunscreen composition. In addition, through combined use of the crosslinked polyether-modified silicone and the silicone oil, such fresh spread at the time of application and smooth texture after the application as to be comparable to those of an aqueous gel can be obtained while excellent stability and a practically sufficient sunscreen effect are maintained.

Any appropriate crosslinked silicone in which silicone chains forming a main chain backbone are crosslinked with each other through a polyether chain may be used as the crosslinked polyether-modified silicone. The polyether chain is preferably a divalent polyoxyalkylene group, more preferably a polyoxyalkylene group having 2 to 8 carbon atoms, still more preferably a polyoxyalkylene group having 2 to 4 carbon atoms (e.g., a polyoxyethylene group, a polyoxypropylene group, or a polyoxybutylene group), even more preferably a polyoxyethylene group. In addition, the silicone chains forming the main chain backbone preferably contain a polydimethylsiloxane backbone, and some of the methyl groups of the polydimethylsiloxane backbone may each be modified with an alkyl group depending on purposes. When some of the methyl groups are each modified with an alkyl group, examples of the alkyl group include linear or branched alkyl groups each having 8 to 20 carbon atoms (e.g., a caprylic group, a lauryl group, and a myristyl group). Those crosslinked polyether-modified silicones may be used alone or in combination.

Specific examples of the crosslinked polyether-modified silicone include a dimethicone/PEG-10/15 crosspolymer, a PEG-15/lauryl dimethicone crosspolymer, a PEG-10/lauryl dimethicone crosspolymer, and a PEG-15/lauryl polydimethylsiloxyethyl dimethicone crosspolymer. Of those, a dimethicone/PEG-10/15 crosspolymer is preferably used. In at least one embodiment of the present invention, the dimethicone/PEG-10/15 crosspolymer may have, for example, a structure represented by the following formula (I):

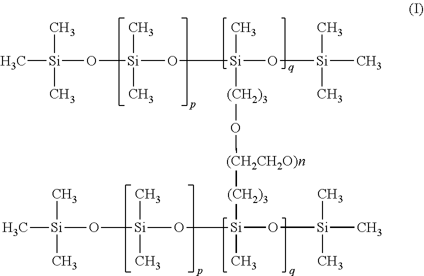

where “n” represents an integer of from 3 to 30, “p” represents an integer of from 1 to 10, and “q” represents an integer of from 1 to 250.

The content ratio of the crosslinked polyether-modified silicone in the sunscreen composition is preferably from 0.01 wt % to 1.0 wt %, more preferably from 0.02 wt % to 0.8 wt %, still more preferably from 0.03 wt % to 0.6 wt %, even more preferably from 0.05 wt % to 0.4 wt %. With the above-mentioned content ratio, a sunscreen composition having high stability and being excellent in feeling of use can be obtained.

<Silicone Oil>

The silicone oil to be used may be a silicone oil that has heretofore been used for cosmetics or the like. Specific examples thereof include cyclomethicone, cyclopentasiloxane, decamethylcyclopentasiloxane, caprylyl methicone, dimethicone (also called dimethylpolysiloxane), a dimethicone/vinyl dimethicone crosspolymer, a dimethicone/phenyl vinyl dimethicone crosspolymer, and diphenylsiloxy phenyl trimethicone. By virtue of the use of the silicone oil, a sunscreen composition having excellent feeling of use and being excellent in stability can be obtained. Those silicone oils may be used alone or in combination.

Of the silicone oils, dimethicone is preferred. When dimethicone and the crosslinked polyether-modified silicone (e.g., dimethicone/PEG-10/15 crosspolymer) are used in combination, such fresh spread at the time of application and smooth texture after the application as to be comparable to those of an aqueous gel can be obtained while a sufficient sunscreen effect is maintained.

The content ratio of the silicone oil in the sunscreen composition is preferably from 3 wt % to 20 wt %, more preferably from 5 wt % to 15 wt %, still more preferably from 8 wt % to 13 wt %.

<Other Oil>

Examples of the oil other than the silicone oil include an ester oil, a natural oil, a hydrocarbon oil, a higher fatty acid, and a higher alcohol.

Examples of the ester oil include isopropyl myristate, octyldodecyl myristate, isopropyl palmitate, butyl stearate, hexyl laurate, myristyl myristate, decyl oleate, hexyldecyl dimethyloctanoate, cetyl lactate, myristyl lactate, lanolin acetate, isocetyl stearate, isocetyl isostearate, cholesteryl 12-hydroxystearate, ethylene glycol di-2-ethylhexanoate, a dipentaerythritol fatty acid ester, an N-alkyl glycol monoisostearate, neopentyl glycol dicaprate, diisostearyl malate, glycerin di-2-heptylundecanoate, trimethylolpropane tri-2-ethylhexanoate, trimethylolpropane triisostearate, pentaerythritol tetra-2-ethylhexanoate, glycerin tri-2-ethylhexanoate, trimethylolpropane triisostearate, cetyl 2-ethylhexanoate, 2-ethylhexyl palmitate, glycerin trimyristate, glyceride tri-2-heptylundecanoate, castor oil fatty acid methyl ester, oleyl oleate, cetostearyl alcohol, acetoglyceride, 2-heptylundecyl palmitate, diisobutyl adipate, 2-octyldodecyl N-lauroyl-L-glutamate, di-2-heptylundecyl adipate, ethyl laurate, di-2-ethylhexyl sebacate, 2-hexyldecyl myristate, 2-hexyldecyl palmitate, 2-hexyldecyl adipate, diisopropyl sebacate, 2-ethylhexyl succinate, triethylhexanoin, ethyl acetate, butyl acetate, amyl acetate, and triethyl citrate.

Examples of the natural oil include avocado oil, camellia oil, turtle oil, macadamia nut oil, kukui nut oil, corn oil, mink oil, olive oil, rapeseed oil, egg-yolk oil, sesame oil, persic oil, wheat germ oil, sasanqua oil, castor oil, linseed oil, safflower oil, cottonseed oil, perilla oil, soybean oil, peanut oil, tea seed oil, kaya oil, rice bran oil, paulownia oil, Japanese tung oil, jojoba oil, germ oil, glyceryl trioctanoate, glyceryl triisopalmitate, and glyceryl tri(caprylate/caprate).

Examples of the hydrocarbon oil include isoalkanes (isoparaffins) each having 8 to 16 carbon atoms, such as isodecane, isododecane, and isohexadecane, a mineral oil, and squalane.

Examples of the higher fatty acid include lauric acid, palmitic acid, stearic acid, isostearic acid, oleic acid, behenic acid, and lanolin acids.

Examples of the higher alcohol include lauryl alcohol, stearyl alcohol, cetyl alcohol, and oleyl alcohol.

The content ratio of the other oil in the sunscreen composition may be set, for example, so that the total blending ratio of the silicone oil and the other oil may be from 10 wt % to 45 wt %, preferably from 12 wt % to 40 wt %, more preferably from 15 wt % to 30 wt %.

<Water>

The water to be used may be water that is generally applicable to cosmetics.

The content ratio of the water in the sunscreen composition is, for example, from 30 wt % to 80 wt %, preferably from 35 wt % to 75 wt %, more preferably from 40 wt % to 65 wt %, still more preferably from 45 wt % to 60 wt %.

<Other Components>

The sunscreen composition may contain any other component than the above-mentioned components as long as the effects of at least one embodiment of the present invention are not impaired. The other component only needs to be a component that may be blended into a cosmetic product, and examples thereof include a thickener and/or an emulsion stabilizer, an emulsifier, a cosmetic component, a moisturizing agent and a texture improver, a pH adjuster, a salt, a fragrance, and a preservative.

The thickener and/or the emulsion stabilizer to be used may be a thickener and/or an emulsion stabilizer that is generally applicable to cosmetics. Specific examples thereof include a carboxyvinyl polymer (carbomer), an acrylates/C10-30 alkyl acrylate crosspolymer, a PVM/MA decadiene crosspolymer, an ammonium acryloyldimethyltaurate/VP copolymer, a PEG-240/HDI copolymer bis-decyltetradeceth-20 ether, sodium polyacrylate, a sodium acrylate/acryloyldimethyl taurate copolymer, a sodium acrylate/sodium acryloyldimethyl taurate copolymer, and a hydroxyethyl acrylate/sodium acryloyldimethyl taurate copolymer.

The content ratio of the thickener and/or the emulsion stabilizer in the sunscreen composition is, for example, from 0.05 wt % to 1 wt %, preferably from 0.1 wt % to 0.5 wt %. In the sunscreen composition according to at least one embodiment of the present invention, the crosslinked polyether-modified silicone can exhibit excellent emulsification stability and an excellent thickening effect, and hence the sunscreen composition can be formulated into a gel even in the case of not further using the thickener and/or the emulsion stabilizer.

The emulsifier to be used may be an emulsifier that is generally applicable to cosmetics. A nonionic surfactant is preferably used, and an ester-type or ether ester-type nonionic surfactant is more preferred. Specific examples thereof include polyoxyethylene fatty acid esters, sorbitan fatty acid esters, sucrose fatty acid esters, polyoxyethylene glycerin fatty acid esters, polyoxyethylene sorbitan fatty acid esters, polyoxyethylene sorbitol fatty acid esters, glycerin fatty acid esters, and polyglycerin fatty acid esters.

Examples of the polyoxyethylene fatty acid esters include polyoxyethylene(6) monolauric acid ester, polyoxyethylene(10) monolauric acid ester, polyoxyethylene(30) monolauric acid ester, polyoxyethylene(1) monostearic acid ester, polyoxyethylene(2) monostearic acid ester, polyoxyethylene (4) monostearic acid ester, polyoxyethylene(10) monostearic acid ester, polyoxyethylene(25) monostearic acid ester, polyoxyethylene (40) monostearic acid ester, polyoxyethylene(45) monostearic acid ester, polyoxyethylene(55) monostearic acid ester, polyoxyethylene(60) monoisostearic acid ester, polyoxyethylene(2) monooleic acid ester, polyoxyethylene(6) monooleic acid ester, and polyoxyethylene(10) monooleic acid ester. In addition, polyoxyethylene di(12-hydroxystearic acid) ester (INCI name: PEG-30 dipolyhydroxystearate) may also be used.

The content ratio of the emulsifier in the sunscreen composition may be preferably from 0.5 wt % to 5 wt %.

Examples of the cosmetic component include niacinamide, comfrey leaf extract, Rosa roxburghii extract, allantoin, Morinda citrifolia juice, Terminalia ferdinandiana fruit extract, Myrciaria dubia fruit extract, ascorbyl glucoside, alpha-glucosyl hesperidin, wild thyme extract, mandarin orange peel extract, soybean seed extract, Scutellaria root extract, xanthophylls, and cyanocobalamin.

Examples of the moisturizing agent and the texture improver include: polyols and polymers thereof, such as glycerin, 1,3-butylene glycol, propylene glycol, 3-methyl-1,3-butanediol, 1,3-propanediol, 2-methyl-1,3-propanediol, trimethylolpropane, 1,2-hexanediol, pentaerythritol, hexylene glycol, diglycerin, polyglycerin, diethylene glycol, polyethylene glycol, dipropylene glycol, polypropylene glycol, and an ethylene glycol-propylene glycol copolymer; glycol alkyl ethers, such as diethylene glycol monoethyl ether (ethoxydiglycol), ethylene glycol monoethyl ether, ethylene glycol monobutyl ether, and diethylene glycol dibutyl ether; esters, such as polyglyceryl-10 eicosanedioate/tetradecanedioate; water-soluble polymers, such as an α-glucan, sodium hyaluronate, collagen, elastin, carageenan, and polyethylene oxide; and glycolipids, such as glucosyl ceramide.

Examples of the pH adjuster include sodium hydroxide, potassium hydroxide, triethanolamine, citric acid, and glycolic acid.

Examples of the preservative include parabens (paraoxybenzoic acid alkyl esters), such as methylparaben, ethylparaben, butylparaben, and benzylparaben, triclosan, and phenoxyethanol. However, it is preferred that the sunscreen composition be substantially free of those preservatives (that is, the content ratio of the preservative in the sunscreen composition is preferably 0.01 wt % or less, more preferably 0.005 wt % or less, still more preferably 0.001 wt % or less). This is because it has been pointed out that those preservatives may also adversely influence coral reefs. For example, the sunscreen composition free of the preservatives can be achieved by utilizing the antimicrobial effect of 1,2-hexanediol or the like given as an example of the moisturizing agent. In this case, the content ratio of the component having an antimicrobial effect, such as 1,2-hexanediol, may be, for example, from 1.0 wt % to 3.0 wt %.

The sunscreen composition maybe produced by any appropriate method. For example, the sunscreen composition may be obtained by heating oily components and aqueous components to any appropriate respective temperatures, and mixing and stirring both types of components.

The SPF value of the sunscreen composition is, for example, 25 or more, preferably 30 or more, more preferably 35 or more. In general, titanium oxide fine particles are more effective for increasing the SPF value than zinc oxide fine particles. However, the sunscreen composition can achieve a practically sufficient SPF value, despite the extremely low blending ratio of the titanium oxide fine particles, as a result of excellent emulsification stability (dispersion stability) and the like.

### EXAMPLES

Now, the present invention is specifically described by way of Examples, but the present invention is by no means limited by these Examples. Measurement methods for characteristics are as described below. Unless otherwise stated, “part(s)” and “%” in Experimental Examples are based on a weight.

Details of components used in the following Experimental Examples are as described below.

Titanium oxide fine particle dispersion: manufactured by Croda, product name: “solaveil XT-300” (titanium oxide content: 47 wt %, solvent: glyceryl tri(caprylate/caprate))

Zinc oxide fine particle dispersion: manufactured by Croda, product name: “solaveil CZ-300” (zinc oxide content: 60 wt %, solvent: glyceryl tri(caprylate/caprate))

PEG-30 dipolyhydroxystearate: manufactured by Croda Japan K.K., product name “CITHROL DPHS”

PPG-15 stearyl ether: manufactured by Croda Japan K.K., product name “SP ARLAMOL PS15E MBAL”

Carbomer: manufactured by Lubrizol Advanced Materials, Inc., product name “Carbopol Ultrez 10 polymer”

Acrylates/C10-30 alkyl acrylate crosspolymer: manufactured by Lubrizol Advanced Materials, Inc., product name “Carbopol Ultrez 21 polymer”

Acrylates/C10-30 alkyl acrylate crosspolymer (self-emulsifying type): manufactured by Lubrizol Advanced Materials, Inc., product name “Pemulen TR-2 Polymeric Emulsifier”

PVM/MA decadiene crosspolymer: manufactured by Ashland Japan Co., Ltd., product name “STABILIZE QM”

Ammonium acryloyldimethyltaurate/VP copolymer: manufactured by Clariant Japan K.K., product name “Aristoflex AVC”

PEG-240/HDI copolymer bis-decyltetradeceth-20 ether: manufactured by ADEKA Corporation, product name “ADEKANOL GT-700”

Dimethicone/PEG-10/15 crosspolymer: manufactured by Shin-Etsu Chemical Co., Ltd., product name “KSG-210” (silicone gel obtained by combining 20% to 30% of a dimethicone/PEG-10/15 crosspolymer with 70% to 80% of dimethicone), used so as to achieve the blending amount of the dimethicone/PEG-10/15 crosspolymer shown in Table 2.

### Experimental Examples 1 to 13

Respective components were mixed in blending amounts shown in Table 1 (total: 100 parts) to provide sunscreen compositions. 0.02 g of each of the resultant sunscreen compositions was applied to a dedicated plastic plate, and dried for 1 hour. After that, the resultant was visually checked for its transparency, and evaluated for a white residue by the following criteria. In addition, as a reference example, a sunscreen composition containing a UV scattering agent and being free of a UV absorber (supplier: GLE LLC, product name: “S Protect Cream”) was used and similarly evaluated for a white residue. The results are shown together in Table 1.

### <<Evaluation Criteria for White Residue 1>>

⊚: Colorless and transparent when applied and spread

o: Showing transparency with whitishness being hardly conspicuous when applied and spread

x: Showing recognizable whiteness even when applied and spread

TABLE 1

| | Reference | Experimental | Experimental | Experimental | Experimental | Experimental | Experimental | Experimental |
|---|---|---|---|---|---|---|---|---|
| | Example 1 | Example 1 | Example 2 | Example 3 | Example 4 | Example 5 | Example 6 | Example 7 |
| PEG-30 dipolyhydroxystearate | — | 2.6 | 2.6 | 2.6 | 2.6 | 2.6 | 2.6 | 2.6 |
| PPG-15 stearyl ether | — | 2 | 2 | 2 | 2 | 2 | 2 | 2 |
| Cyclopentasiloxane | — | 6 | 6 | 6 | 6 | 6 | 6 | 6 |
| Isohexadecane | — | 5 | 5 | 5 | 5 | 5 | 5 | 5 |
| Cetyl ethylhexanoate | — | 8 | 8 | 8 | 8 | 8 | 8 | 8 |
| Glycerin | — | 4 | 4 | 4 | 4 | 4 | 4 | 4 |
| solaveil CZ-300 | — | 50 (30) | 30 (18) | 15 (9) | 10 (6) | 5 (3) | 2 (1.2) | 1 (0.6) |
| (ZnO content) | | | | | | | | |
| Water | — | Balance | Balance | Balance | Balance | Balance | Balance | Balance |
| White residue | X | ⊚ | ⊚ | ⊚ | ⊚ | ⊚ | ⊚ | ⊚ |

| | Experimental | Experimental | Experimental | Experimental | Experimental | Experimental |
|---|---|---|---|---|---|---|
| | Example 8 | Example 9 | Example 10 | Example 11 | Example 12 | Example 13 |
| PEG-30 dipolyhydroxystearate | 2.6 | 2.6 | 2.6 | 2.6 | 2.6 | 2.6 |
| PPG-15 stearyl ether | 2 | 2 | 2 | 2 | 2 | 2 |
| Cyclopentasiloxane | 6 | 6 | 6 | 6 | 6 | 6 |
| Isohexadecane | 5 | 5 | 5 | 5 | 5 | 5 |
| Cetyl ethylhexanoate | 8 | 8 | 8 | 8 | 8 | 8 |
| Glycerin | 4 | 4 | 4 | 4 | 4 | 4 |
| solaveil XT-300 | 1 (0.47) | 2 (0.94) | 5 (2.4) | 15 (7.1) | 20 (9.4) | 20 (9.4) |
| (TiO₂ content) | | | | | | |
| solaveil CZ-300 | 20 (12) | 20 (12) | 15 (9) | 5 (3) | 2 (1.2) | 1 (0.6) |
| (ZnO content) | | | | | | |
| Water | Balance | Balance | Balance | Balance | Balance | Balance |
| White residue | ◯ | X | X | X | X | X |

As shown in Table 1, the sunscreen compositions of Experimental Examples 9 to 13, in each of which the content ratio of the titanium oxide fine particles was more than 0.8 wt %, each generated a white residue. Meanwhile, the sunscreen compositions of Experimental Examples 1 to 8, in each of which the content ratio of the titanium oxide fine particles was 0.8 wt % or less, were each suppressed in white residue, and the effect was remarkable in each of the sunscreen compositions of Experimental Examples 1 to 7 free of the titanium oxide fine particles.

### Experimental Examples 14 to 20

Respective components were mixed in blending amounts shown in Table 2 (total: 100 parts) in accordance with a conventional method to provide water-in-oil sunscreen compositions. In Table 2, the “gel” formulation means physical properties of having such a high viscosity as to be able to exhibit a certain level of shape retainability in a stress-free state, and changing into a sol through a reduction in viscosity when subjected to an external force. The state of each of the resultant sunscreen compositions after 72 hours from its preparation was observed, and its stability was evaluated by the following criteria. The results are shown together in Table 2.

### <<Evaluation Criteria for Stability>>

o: None of separation, aggregation and unevenness of resin, and discoloration is found.

x: Any one of separation, aggregation and unevenness of resin, and discoloration is found.

Ten panelists each applied and spread 0.5 g of the sunscreen composition of Experimental Example 20, which showed satisfactory stability in the stability evaluation, over a forearm with a finger, and evaluated a white residue and feeling of use by the following evaluation criteria. The results are shown together in Table 2.

### <<Evaluation Criteria for Feeling of Use 1>>

o: Freshness and spreadability at the time of application are satisfactory.

x: Freshness and spreadability at the time of application are unsatisfactory.

### <<Evaluation Criteria for Feeling of Use 2>>

o: The feeling of touch after application is smooth.

x: The feeling of touch after application involves stickiness.

### <<Evaluation Criteria for White Residue 2>>

o: No white residue is visually recognized.

x: A white residue is visually recognized.

TABLE 2

| | Experimental | Experimental | Experimental | Experimental | Experimental | Experimental | Experimental |
|---|---|---|---|---|---|---|---|
| | Example 14 | Example 15 | Example 16 | Example 17 | Example 18 | Example 19 | Example 20 |
| CZ-300 | 30 | 30 | 30 | 30 | 30 | 30 | 30 |
| Dimethicone | 10 | 10 | 10 | 10 | 10 | 10 | 10 |
| Kukui nut oil | 0.1 | 0.1 | 0.1 | 0.1 | 0.1 | 0.1 | 0.1 |
| NaCl | 0.2 | 0.2 | 0.2 | 0.2 | 0.2 | 0.2 | 0.2 |
| 1,2-Hexanediol | 1.2 | 1.2 | 1.2 | 1.2 | 1.2 | 1.2 | 1.2 |
| Allantoin | 0.1 | 0.1 | 0.1 | 0.1 | 0.1 | 0.1 | 0.1 |
| Ascorbyl glucoside | 2.0 | 2.0 | 2.0 | 2.0 | 2.0 | 2.0 | 2.0 |
| Alpha-glucosyl hesperidin | 0.1 | 0.1 | 0.1 | 0.1 | 0.1 | 0.1 | 0.1 |
| Carbomer | 0.3 | — | — | — | — | — | — |
| Acrylates/C10-30 | — | 0.3 | — | — | — | — | — |
| alkyl acrylate | | | | | | | |
| crosspolymer | | | | | | | |
| Acrylates/C10-30 | — | — | 0.3 | — | — | — | — |
| alkyl acrylate | | | | | | | |
| crosspolymer (self- | | | | | | | |
| emulsifying type) | | | | | | | |
| PVM/MA decadiene | — | — | — | 0.3 | — | — | — |
| crosspolymer | | | | | | | |
| Ammonium | — | — | — | — | 0.3 | — | — |
| acryloyldimethyltaurate/ | | | | | | | |
| VP copolymer | | | | | | | |
| PEG-240/HDI copolymer | — | — | — | — | — | 0.6 | — |
| bis-decyltetradeceth-20 ether | | | | | | | |
| Dimethicone/PEG-10/15 | — | — | — | — | — | — | 0.1 |
| crosspolymer | | | | | | | |
| KOH | 0.12 | 0.12 | 0.12 | — | — | — | — |
| Water | Balance | Balance | Balance | Balance | Balance | Balance | Balance |
| Formulation | Gel | Gel | Gel | Gel | Gel | Gel | Gel |
| Stability | X | X | X | X | X | X | ◯ |
| Feeling of use 1 | — | — | — | — | — | — | ◯ |
| Feeling of use 2 | — | — | — | — | — | — | ◯ |
| White residue | — | — | — | — | — | — | ◯ |

As shown in Table 2, through combined use of the zinc oxide fine particles, the crosslinked polyether-modified silicone, and the silicone oil, a sunscreen composition suppressed in white residue, and excellent in stability and feeling of use can be obtained.

The sunscreen composition according to at least one embodiment of the present invention can be suitably utilized in the field of cosmetics.

According to at least one embodiment of the present invention, the sunscreen composition free of the above-mentioned prohibited components, the sunscreen composition being ameliorated in terms of the problems with feeling of use and the problem of a white residue, is provided.

Many other modifications will be apparent to and be readily practiced by those skilled in the art without departing from the scope and spirit of the invention. It should therefore be understood that the scope of the appended claims is not intended to be limited by the details of the description but should rather be broadly construed.

What is claimed is:

1. A sunscreen composition, comprising:
zinc oxide fine particles;
a crosslinked polyether-modified silicone; and
a silicone oil,
wherein a content ratio of the zinc oxide fine particles is from 8 wt % to 40 wt %,
wherein the sunscreen composition is free of titanium oxide fine particles, or contains titanium oxide fine particles at a content ratio of 0.8 wt % or less, and
wherein the sunscreen composition is free of a UV absorber.

2. The sunscreen composition according to claim 1, wherein a content ratio of the crosslinked polyether-modified silicone is from 0.01 wt % to 1 wt %.

3. The sunscreen composition according to claim 1, wherein a content ratio of the silicone oil is from 5 wt % to 15 wt %.

4. The sunscreen composition according to claim 1, wherein the crosslinked polyether-modified silicone is a dimethicone/PEG-10/15 crosspolymer.

5. The sunscreen composition according to claim 1, wherein the silicone oil is dimethicone.

6. The sunscreen composition according to claim 1, wherein the sunscreen composition is substantially free of the titanium oxide fine particles.

7. The sunscreen composition according to claim 1, wherein the sunscreen composition is substantially free of paraoxybenzoic acid alkyl esters, phenoxyethanol, and triclosan.

8. The sunscreen composition according to claim 1,
wherein the content ratio of the zinc oxide fine particles is from 15 wt % to 30 wt %,
wherein the crosslinked polyether-modified silicone is a dimethicone/PEG-10/15 crosspolymer,
wherein a content ratio of the dimethicone/PEG-10/15 crosspolymer is from 0.01 wt % to 1 wt %,
wherein the silicone oil is dimethicone, and
wherein a blending ratio of the dimethicone is from 5 wt % to 15 wt %.

9. The sunscreen composition according to claim 8, wherein the sunscreen composition is substantially free of paraoxybenzoic acid alkyl esters, phenoxyethanol, and triclosan.

10. The sunscreen composition according to claim 8, wherein the sunscreen composition is free of the titanium oxide fine particles.

11. The sunscreen composition according to claim 10, wherein the sunscreen composition is substantially free of paraoxybenzoic acid alkyl esters, phenoxyethanol, and triclosan.
